(19) United States
(12) Patent Application Publication
(10) Pub. No.: US 2026/0087770 A1
(43) Pub. Date: Mar. 26, 2026

(54) METHOD AND SYSTEM FOR ASSESSING CONTAMINATION LEVEL OF NON-AQUEOUS PHASE LIQUIDS (NAPL) IN GROUNDWATER

(71) Applicants: BCEG ENVIRONMENT REMEDIATION CO., LTD., Beijing (CN); Beijing Construction Engineering Ecological Restoration Laboratory Technology Co., LTD, Beijing (CN)

(72) Inventors: Shupeng LI, Beijing (CN); Beili WANG, Beijing (CN); Lili GUO, Beijing (CN); Yunxiao WEI, Beijing (CN); Jiaming ZHANG, Beijing (CN); Xu YANG, Beijing (CN); Jinmei XUE, Beijing (CN); Qianwu WANG, Beijing (CN)

(21) Appl. No.: 19/405,312

(22) Filed: Dec. 1, 2025

Related U.S. Application Data
(63) Continuation of application No. PCT/CN2023/127816, filed on Oct. 30, 2023.

(30) Foreign Application Priority Data
Jun. 2, 2023 (CN) 202310645177.6

(51) Int. Cl. G06V 10/44; G01N 33/18; G06T 5/70; G06T 7/10; G06V 10/74

(52) U.S. Cl. CPC G06V 10/44; G01N 33/18; G06T 5/70; G06T 7/10; G06V 10/74

(57) ABSTRACT

A method and system for assessing contamination level of non-aqueous phase liquids (NAPL) in groundwater based on image learning are provided herein. A water image data of groundwater monitoring wells is acquired, processed, and extracted to give image features. Types and concentrations of the NAPLs in groundwater are obtained based on monitoring data of groundwater monitoring wells collected within a preset period, and matched with the image features of the water image data to construct a feature dataset. A groundwater NAPL contamination database is established based on the feature dataset in combination with local hydrogeological information. A groundwater NAPL contamination level assessment model is constructed based on deep learning, and trained by utilizing the groundwater NAPL contamination database. The NAPL contamination level of water in the groundwater monitoring wells is determined to issue the contamination early warning.

### CROSS-REFERENCE TO RELATED APPLICATIONS

This application is a continuation of International Patent Application No. PCT/CN2023/127816, filed on Oct. 30, 2023, which claims the benefit of priority from Chinese Patent Application No. 202310645177.6, filed on Jun. 2, 2023. The content of the aforementioned application, including any intervening amendments thereto, is incorporated herein by reference in its entirety.

### TECHNICAL FIELD

This application relates to monitoring and assessment of groundwater contamination, and more particularly to a method and system for assessing contamination level of non-aqueous phase liquids (NAPL) in groundwater.

### BACKGROUND

Groundwater contamination severely threatens the water supply safety for urban residents. Assessment of groundwater contamination plays a critical role in the investigation and remediation of the groundwater contamination, and can provide a scientific basis for prevention and control of the groundwater contamination in research areas.

Upon entering a soil aquifer, non-aqueous phase liquids (NAPLs) cannot be mixed with water, but instead persist in liquid or gaseous form in soil and groundwater. NAPLs can be mainly classified into light non-aqueous phase liquids (LNAPLs) (such as petroleum hydrocarbons and benzene-based compounds) and dense non-aqueous phase liquids (DNAPLs) (such as chlorinated hydrocarbons and polychlorinated biphenyls). Following a leak, NAPLs can infiltrate vertically through the soil into the groundwater, and then spread radially with the groundwater flow, forming a contamination plume. The assessment of NAPL contamination in groundwater is complicated by several factors such as NAPLs' hydrophobicity, differential migration behaviors of complex pollutants, subsurface heterogeneity, and dynamic interactions between surface water and groundwater in a contaminated site. Consequently, there is an urgent need to achieve precise assessment and early warning of groundwater NAPL contamination level.

### SUMMARY

In view of this, the present disclosure provides a method and system for assessing contamination level of non-aqueous phase liquids (NAPL) in groundwater based on image learning.

In a first aspect, the present disclosure provides a method for assessing contamination level of non-aqueous phase liquids (NAPL) in groundwater based on image learning, comprising:
(S1) acquiring water image data of groundwater monitoring wells within a research area; preprocessing the water image data; and extracting image features from a preprocessed water image data;
(S2) obtaining types and concentrations of NAPLs in groundwater based on monitoring data of the groundwater monitoring wells collected within a preset period;
(S3) matching the image features with the types and concentrations of the NAPLs to construct a feature dataset; and establishing a groundwater NAPL contamination database based on the feature dataset in combination with local hydrogeological information;
(S4) constructing a groundwater NAPL contamination level assessment model based on deep learning; training the groundwater NAPL contamination level assessment model by utilizing the groundwater NAPL contamination database; determining a NAPL contamination level of water in the groundwater monitoring wells based on a trained groundwater NAPL contamination level assessment model; and issuing an early warning based on the NAPL contamination level.

In an embodiment, step (S1) comprises:
obtaining, by an image sensor deployed in the groundwater monitoring wells, the water image data over a preset time interval;
subjecting the water image data to filtering for denoising, enhancement and partitioning to generate a plurality of image patches;
calculating an information entropy of each of the plurality of image patches;
generating a mask image of the water image data based on information entropies of the plurality of image patches;
performing an image segmentation based on the mask image of the water image data to obtain the preprocessed water image data;
inputting the preprocessed water image data into a cross stage partial network (CSPNET) for feature characterization to generate a feature map;
introducing the feature map into an atrous spatial pyramid pooling (ASPP) module for multi-scale feature extraction by using dilated convolutions with different dilation rates to generate a multi-scale feature map;
transforming the multi-scale feature map into a channel descriptor through average pooling;
calculating a channel attention weight of the multi-scale feature map through two convolutional layers and sigmoid and rectified linear unit (ReLU) functions;
multiplying the multi-scale feature map by the channel attention weight to generate an attention map; and
performing element-wise multiplication between the attention map and the feature map to obtain the image features.

In an embodiment, steps (S2-S3) comprises:
reading historical monitoring data of the groundwater monitoring wells within the research area;
determining whether each of the groundwater monitoring wells whether is valid based on the historical monitoring data, and removing invalid groundwater monitoring wells;
acquiring monitoring data of valid groundwater monitoring wells within the preset period;
performing data cleaning on the monitoring data of the valid groundwater monitoring wells to obtain the types and concentrations of the NAPLs in groundwater;
integrating the image features with the types and concentrations of the NAPLs in groundwater to construct the feature dataset;
collecting flow direction and velocity of the groundwater, surface water-groundwater interaction frequency and a stratigraphic information in the research area to construct a hydrogeological dataset;
combining the hydrogeological dataset with the feature dataset to generate a combined feature data;
establishing the groundwater NAPL contamination database;
structuring the combined feature data, and storing a structured feature data into the groundwater NAPL contamination database; and
performing data management of groundwater NAPL contamination in the research area, and updating and optimization of the groundwater NAPL contamination level assessment model based on the groundwater NAPL contamination database.

In an embodiment, steps of constructing the groundwater NAPL contamination level assessment model based on deep learning and training the groundwater NAPL contamination level assessment model by utilizing the groundwater NAPL contamination database comprise:
constructing the groundwater NAPL contamination level assessment model based on deep learning;
extracting the feature dataset from the groundwater NAPL contamination database;
acquiring criteria for assessing contamination levels of each of the types corresponding to NAPL contamination in a preset retrieval space by means of big data;
selecting a criterion with a highest occurrence frequency from testing data to construct an assessing system for the contamination levels of each of the types;
performing contamination level assessment based on the assessing system and a part of the monitoring data of the groundwater monitoring wells corresponding to the feature dataset;
respectively setting contamination level annotations for the image features to give an annotated feature dataset;
training the groundwater NAPL contamination level assessment model based on the annotated feature dataset until training iterations reaches a preset value, thereby outputting a trained groundwater NAPL contamination assessment model;
inputting the image features of the water image data and the local hydrogeological information of the research area into the trained groundwater NAPL contamination assessment model to identify types and concentration information corresponding to the image features of each of the groundwater monitoring wells; and
acquiring a current spatial distribution of the NAPLs and the groundwater NAPL contamination levels within the research area.

In an embodiment, before generating the early warning based on the groundwater NAPL contamination level, the method further comprises:
dividing the research area into a plurality of sub-areas based on distribution of the groundwater monitoring wells;
obtaining types and historical concentrations of the NAPLs in each of the plurality of sub-areas within the preset period;
generating a concentration time series of corresponding to the types based on the historical concentrations;
tracing contamination sources from different types based on the concentration time series in each of the plurality of sub-areas;
marking each of the plurality of sub-areas contaminated by the contamination sources, and setting labels for the plurality of sub-areas according to the types of the contamination sources;
extracting a concentration change over the preset time interval based on the concentration time series and setting different change thresholds for labeled sub-areas and unlabeled sub-areas;
determining whether each of the plurality of sub-areas is labeled and reading a change threshold based on a determination result;
in a case that the concentration change exceeds the change threshold, if a sub-area is not labeled, acquiring environmental features and climate features of the sub-area within a next preset period; and if a sub-area is labeled, acquiring environmental features, climate features and contamination source operational features of the sub-area over the next preset period;
acquiring an average concentration change of the plurality of sub-areas over the preset time interval; and
setting the average concentration change as a contamination change reference value within the preset period for each of the plurality of sub-areas.

In an embodiment, the step of generating the early warning based on the groundwater NAPL contamination level comprises:
acquiring current types and concentrations of the NAPLs in each of the plurality of sub-areas based on a current spatial distribution of the NAPLs in the research area, and correspondingly comparing the current types and concentrations with historical types and concentrations of the NAPLs within a previous preset period;
if the types of the NAPLs in a sub-area change, or the concentration change of the NAPLs in a sub-area is greater than the contamination change reference value, generating an early warning of the sub-area, and visually displaying the early warning and the current spatial distribution of the NAPLs;
if a sub-area is not labeled, acquiring environmental features and climate features of the sub-area within a next preset period; if a sub-area is labeled, acquiring environmental features, climate features and contamination source operational features of the sub-area over the next preset period; and calculating a similarity between acquired features and the contamination impact features of each of the plurality of sub-areas based on a Manhattan distance; and
if the Manhattan distance is not greater than a preset distance threshold, confirming that the similarity satisfies a preset requirement and generating a contamination early warning for a corresponding sub-area; and generating and issuing a contamination emergency measure for the corresponding sub-area by means of big data based on the contamination early warning.

In a second aspect, a system for assessing contamination levels of non-aqueous phase liquid (NAPL) in groundwater based on image learning, comprising:
a memory; and
a processor;
wherein the memory is configured to store a program of a method for assessing contamination levels of non-aqueous phase liquid (NAPL) in groundwater based on image learning; and
the processor is configured to execute the program stored in the memory to implement the aforementioned method through steps of:
(S1) acquiring water image data of groundwater monitoring wells within a research area; preprocessing the water image data; and extracting image features from a preprocessed water image data;
(S2) obtaining types and concentrations of NAPLs in groundwater based on monitoring data of the groundwater monitoring wells collected within a preset period;
(S3) matching the image features with the types and concentrations of the NAPLs to construct a feature dataset; and establishing a groundwater NAPL contamination database based on the feature dataset in combination with local hydrogeological information;
(S4) constructing a groundwater NAPL contamination level assessment model based on deep learning; training the groundwater NAPL contamination level assessment model by utilizing the groundwater NAPL contamination database; determining a NAPL contamination level of water in the groundwater monitoring wells based on a trained groundwater NAPL contamination level assessment model; and issuing an early warning based on the NAPL contamination level.

The present disclosure provides the method and system for assessing contamination level of non-aqueous phase liquids (NAPL) in groundwater based on image learning. Specifically, the method and system are performed through the following steps. The water image data of the groundwater monitoring wells is acquired, processed, and extracted to give the image features of the water image data. The types and concentrations of the NAPLs in groundwater are obtained based on the monitoring data of the groundwater monitoring wells collected within a preset period, and matched with the image features of the water image data to construct the feature dataset. The groundwater NAPL contamination database is established based on the feature dataset in combination with local hydrogeological information. The groundwater NAPL contamination level assessment model is constructed based on deep learning, and trained by utilizing the groundwater NAPL contamination database. The NAPL contamination level of water in the groundwater monitoring wells is determined to issue the contamination early warning. The present disclosure achieves a quick assessment of the NAPL contamination level by establishing a correlation between the water image data, and the types and concentrations of the NAPLs, so as to provide a data support for prevention and control of the groundwater contamination in the research area.

### BRIEF DESCRIPTION OF THE DRAWINGS

FIG. 1 is a flow diagram of a method for assessing contamination level of non-aqueous phase liquids (NAPL) in groundwater based on image learning according to an embodiment of the present disclosure;

FIG. 2 is a flow diagram of a method for constructing a groundwater NAPL contamination level assessment model based on deep learning according to an embodiment of the present disclosure;

FIG. 3 is a flow diagram of a method for issuing an early warning based on the NAPL contamination level according to an embodiment of the present disclosure; and

FIG. 4 schematically shows a system for assessing contamination level of non-aqueous phase liquids (NAPL) in groundwater according to an embodiment of the present disclosure.

### DETAILED DESCRIPTION OF EMBODIMENTS

In order to make the above objects, features and advantages of the present disclosure clearer, the present disclosure will be further detailly described below with reference to the accompanying drawings and embodiments. It should be noted that the embodiments of the present disclosure and the features of the embodiments can be combined with each other as long as there is no contradiction.

Many specific details are provided below to facilitate understanding of the present disclosure. Obviously, the present disclosure can be implemented in many other ways different from those described herein. Therefore, the embodiments described below are not intended to limit this present disclosure.

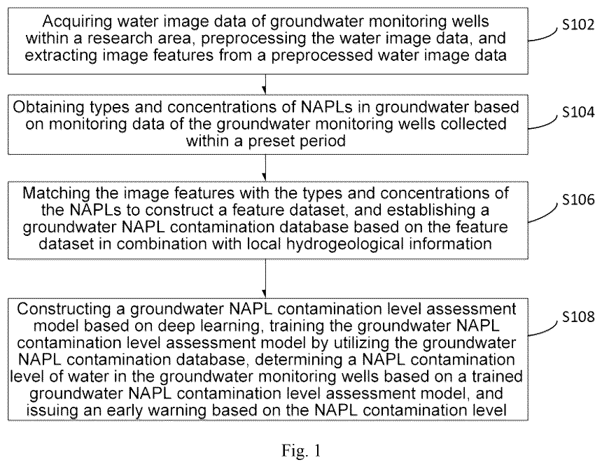

A flow diagram of a method for assessing contamination level of non-aqueous phase liquids (NAPL) in groundwater according to an embodiment of the present disclosure is shown in FIG. 1.

Referring to FIG. 1, the present disclosure provides a method for assessing contamination level of non-aqueous phase liquids (NAPL) in groundwater. The method provided herein is performed through the following steps.

(S102) Water image data of groundwater monitoring wells within a research area is acquired and processed to give a preprocessed water image data, and image features are extracted from the preprocessed water image data.

(S104) Types and concentrations of NAPLs in groundwater are obtained based on monitoring data of the groundwater monitoring wells collected within a preset period are obtained.

(S106) The image features are matched with the types and concentrations of the NAPLs to construct a feature dataset, and a groundwater NAPL contamination database is established based on the feature dataset in combination with local hydrogeological information.

(S108) A groundwater NAPL contamination level assessment model is constructed based on deep learning, and trained by utilizing the groundwater NAPL contamination database to determine a NAPL contamination level of water in the groundwater monitoring wells and issue an early warning based on the NAPL contamination level.

It should be noted that, after obtaining, by an image sensor deployed in the groundwater monitoring wells, the water image data over a preset time interval, the water image data is subjected to filtering for denoising, enhancing and partitioning to generate a plurality of image patches.

In some embodiments, the water image data is enhanced through a generative adversarial network. In the generative adversarial network, an original water image data is input into a generator, and subjected to a series of operational steps to output an enhanced image and a difference between the enhanced image and a reference image, followed by adjusting parameters of the generator and sending results back to the generator. Thus, the generator is constantly improved, and discrimination ability of a corresponding discriminator is further adjusted until an equilibrium between the generator and the corresponding discriminator is reached, thereby enhancing the water information through the generative adversarial network.

After filtering, denoising, and enhancing, the water image data is partitioned to the plurality of image patches. An information entropy of each of the plurality of image patches is calculated to characterize feature abundance in the water image data. A mask image of the water image data is generated based on information entropies of the plurality of image patches. An image segmentation is performed based on the mask image of the water image data to obtain the preprocessed water image data. The preprocessed water image data is input into a cross stage partial network (CSPNET) for feature characterization to generate a feature map. The CSPNET can acquire more abundant and stronger feature characterization, simultaneously reducing computational complexity. The feature map is introduced into an atrous spatial pyramid pooling (ASPP) module for multi-scale feature extraction by using dilated convolutions with different dilation rates to generate a multi-scale feature map. The introduction of dilated convolutions effectively increases a receptive field to encompass more contextual information without increasing computational complexity.

The multi-scale feature map is transformed into a channel descriptor f through average pooling, satisfying the following relationship:

$$f = \frac{1}{MN} {\sum }_{i}^{M} {\sum }_{j}^{N} {T}_{c} ( i , j ) ,$$

where MN represents a size of a feature map; and T_{c}(i,j) represents a c-th channel T_{c} at a point of (i,j).

In some embodiments, a channel attention weight q of the multi-scale feature map is calculated through two convolutional layers and sigmoid and rectified linear unit (ReLU) functions, where q satisfies the following relationship:

$$q = Sigmoid ( conv ( Relu ( conv ( f ) ) ) ) ,$$

where conv represents a convolution computation.

The multi-scale feature map is multiplied by the channel attention weight to generate an attention map. An element-wise multiplication between the attention map and the feature map is performed to obtain the image features.

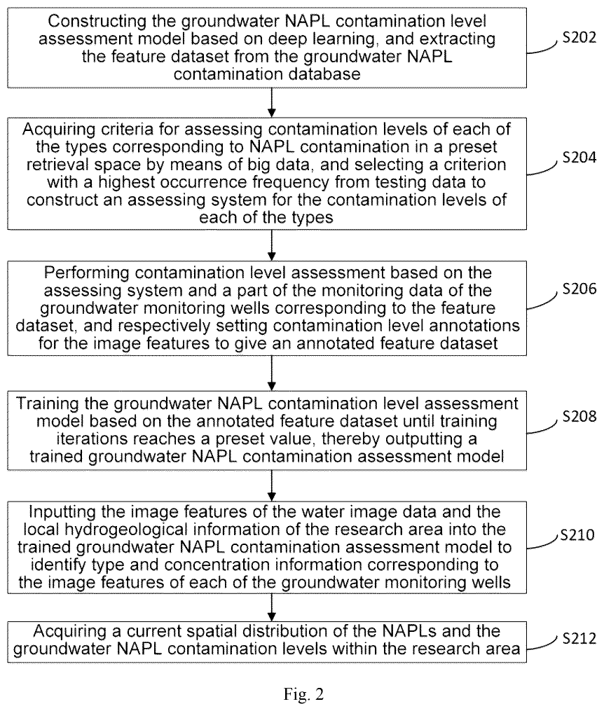

A flow diagram of a method for constructing a groundwater NAPL contamination level assessment model based on deep learning according to an embodiment of the present disclosure is shown in FIG. 2.

According to the embodiment of FIG. 2, the method for constructing the groundwater NAPL contamination level assessment model based on deep learning and training the groundwater NAPL contamination level assessment model by utilizing the groundwater NAPL contamination database is performed through the following steps.

(S202) The groundwater NAPL contamination level assessment model is constructed based on deep learning, and the feature dataset is extracted from the groundwater NAPL contamination database.

(S204) Criteria for assessing contamination levels of each of the types corresponding to NAPL contamination are acquired in a preset retrieval space by means of big data, and a criterion with a highest occurrence frequency is selected from testing data to construct a system for an assessing system for the contamination levels of each of the types.

(S206) A contamination level assessment is performed based on the assessing system and a part of the monitoring data of the groundwater monitoring wells corresponding to the feature dataset, and contamination level annotations are set for the image features to give an annotated feature dataset.

(S208) The groundwater NAPL contamination level assessment model is trained based on the annotated feature dataset until training iterations reaches a preset value, thereby outputting a trained groundwater NAPL contamination assessment model.

(S210) The image features of the water image data and the local hydrogeological information of the research area are input into the trained groundwater NAPL contamination assessment model to identify the type and concentration information corresponding to the image features of each of the groundwater monitoring wells.

(S212) A current spatial distribution of the NAPLs and the groundwater NAPL contamination levels within the research area are acquired.

It should be noted that, the method based on deep learning include backpropagation (BP) neural networks and support vector machines (SVM), and the preset retrieval space includes relevant literature databases and expert experience bases.

Historical monitoring data of the groundwater monitoring wells within the research area is read to determine whether each of the groundwater monitoring wells is valid, and invalid groundwater monitoring wells are removed. The monitoring data of valid groundwater monitoring wells is acquired within the preset period and performed a data cleaning to obtain the types and concentrations of the NAPLs in groundwater. The image features are integrated with the types and concentrations of the NAPLs in groundwater to construct the feature dataset. Flow direction and velocity of the groundwater, surface water-groundwater interaction frequency and a stratigraphic information in the research area are collected to construct a hydrogeological dataset, followed by combining the hydrogeological dataset with the feature dataset to generate a combined feature data. The groundwater NAPL contamination database is established. The combined feature data is structured to obtain a structured feature data. The structured feature data is stored into the groundwater NAPL contamination database. Data management of groundwater NAPL contamination in the research area is performed, and updating and optimization of the groundwater NAPL contamination level assessment model are also performed based on the groundwater NAPL contamination database.

It should be noted that, the image features in the feature dataset are clustered according to the types of the NAPLs. A scatter distribution of the image features in a feature space is obtained for each of the types of the NAPLs. A statistical analysis is performed on the image features under the same type of the NAPLs to obtain the image features with the highest occurrence frequency. A similarity is calculated based on the image features to obtain a preset number of image features that satisfy a preset standard to construct the feature dataset. Corresponding positions of the image features of the feature dataset in the scatter distribution are obtained to give cluster centroids of the image features for the types of the NAPLs. A distance threshold corresponding to each of the types of the NAPLs is obtained based on an average distance between other points in the scatter distribution and each of the cluster centroids. A distance between the image features of the plurality of image patches in the water image data and each of the cluster centroids of the image features for each of the types of the NAPLs is compared with the distance threshold corresponding to this type. If the distance is less than the distance threshold, it is proved that the water is contaminated by this type of the NAPLs.

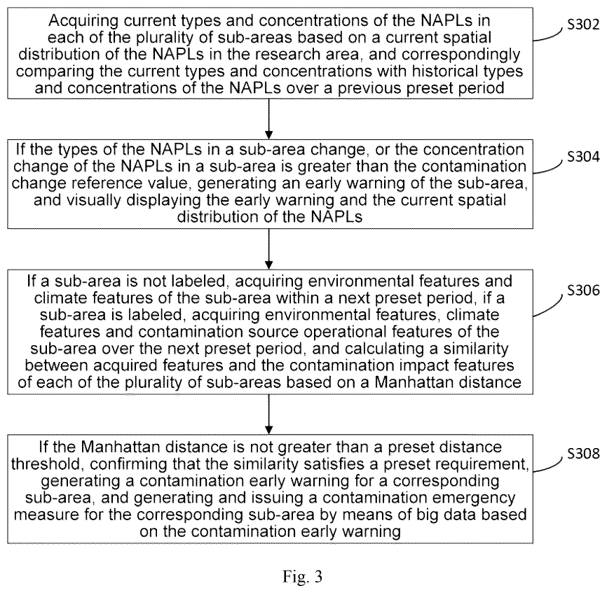

A flow diagram of a method for issuing an early warning based on the NAPL contamination level according to an embodiment of the present disclosure is shown in FIG. 3.

According to the embodiment of FIG. 3, the method for issuing an early warning based on the NAPL contamination level is performed through the following steps.

(S302) Current types and concentrations of the NAPLs in each of the plurality of sub-areas are acquired based on a current spatial distribution of the NAPLs in the research area, and the current types and concentrations are correspondingly compared with historical types and concentrations of the NAPLs over a previous preset period.

(S304) If the types of the NAPLs in a sub-area change, or the concentration change of the NAPLs in a sub-area is greater than the contamination change reference value, the early warning of the sub-area is generated, and the early warning and the current spatial distribution of the NAPLs are visually displayed.

(S306) If a sub-area is not labeled, environmental features and climate features of each of the sub-area are acquired within a next preset period, and if a sub-area is labeled, environmental features, climate features and contamination source operational features of the sub-area are acquired over the next preset period, and a similarity between acquired features and the contamination impact features of each of the plurality of sub-areas is calculated based on a Manhattan distance.

(S308) If the Manhattan distance is not greater than a preset distance threshold, it is confirmed that the similarity satisfies a preset requirement, and generating a contamination early warning for a corresponding sub-area is generated, and a contamination emergency measure for the corresponding sub-area is generated and issued by means of big data based on the contamination early warning.

It should be noted that the research area is divided into a plurality of sub-areas based on distribution of the groundwater monitoring wells. Types and historical concentrations of the NAPLs in each of the plurality of sub-areas are obtained within the preset period to generate a concentration time series corresponding to the types. The contamination sources are traced from different types traced based on the concentration time series in each of the plurality of sub-areas. Each of the plurality of sub-areas contaminated by the contamination sources is marked, and is set labels according to the types of the contamination sources. A concentration change over the preset time interval is extracted based on the concentration time series of the types to set different change thresholds for labeled sub-areas and unlabeled sub-areas. It is determined whether each of the plurality of sub-areas is labeled to give a labeled sub-area or an unlabeled sub-area, followed by reading a change threshold based on a determination result.

In a case that the concentration change exceeds the change threshold, if a sub-area is not labeled, environmental features and climate features of the sub-area are acquired as contamination impact features of the sub-area; and if a sub-area is labeled, environmental features, climate features and contamination source operational features of the sub-area are acquired as the contamination impact features of the sub-area. The contamination source operational features include a production plan and production quantity of a factory, etc. Meanwhile, an average concentration change of the plurality of sub-areas over the preset time interval is acquired in the sub-area to be set as a contamination change reference value within the preset period for each of the plurality of sub-areas.

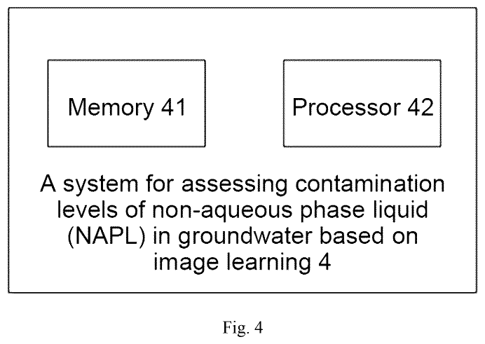

A system for assessing contamination levels of non-aqueous phase liquid (NAPL) in groundwater based on image learning according to an embodiment of the present disclosure is schematically shown in FIG. 4.

In a second aspect, the present disclosure provides a system for assessing contamination levels of non-aqueous phase liquid (NAPL) in groundwater based on image learning 4, including a memory 41 and a processor 42. The memory 41 is configured to store a program. The processor 42 is configured to execute the program stored in the memory 41 to perform the following steps.

The water image data of the groundwater monitoring wells is acquired, processed, and extracted to give the image features of the water image data. The types and concentrations of the NAPLs in groundwater are obtained based on the monitoring data of the groundwater monitoring wells collected within a preset period, and matched with the image features of the water image data to construct the feature dataset. The groundwater NAPL contamination database is established based on the feature dataset in combination with local hydrogeological information. The groundwater NAPL contamination level assessment model is constructed based on deep learning, and trained by utilizing the groundwater NAPL contamination database. The NAPL contamination level of water in the groundwater monitoring wells is determined to issue the contamination early warning.

It should be noted that, after obtaining, by an image sensor deployed in the groundwater monitoring wells, the water image data over a preset time interval, the water image data is subjected to filtering for denoising, enhancing and partitioning to generate a plurality of image patches.

In some embodiments, the water image data is enhanced through a generative adversarial network. In the generative adversarial network, an original water image data is input into a generator, and subjected to a series of operational steps to output an enhanced image and a difference between the enhanced image and a reference image, followed by adjusting parameters of the generator and sending results back to the generator. Thus, the generator is constantly improved, and discrimination ability of a corresponding discriminator is further adjusted until an equilibrium between the generator and the corresponding discriminator is reached, thereby enhancing the water information through the generative adversarial network.

After filtering, denoising, and enhancing, the water image data is partitioned to the plurality of image patches. An information entropy of each of the plurality of image patches is calculated to characterize feature abundance in the water image data. A mask image of the water image data is generated based on information entropies of the plurality of image patches. An image segmentation is performed based on the mask image of the water image data to obtain the preprocessed water image data. The preprocessed water image data is input into a cross stage partial network (CSPNET) for feature characterization to generate a feature map. The CSPNET can acquire more abundant and stronger feature characterization, simultaneously reducing computational complexity. The feature map is introduced into an atrous spatial pyramid pooling (ASPP) module for multi-scale feature extraction by using dilated convolutions with different dilation rates to generate a multi-scale feature map. The introduction of dilated convolutions effectively increases a receptive field to encompass more contextual information without increasing computational complexity.

The multi-scale feature map is transformed into a channel descriptor f through average pooling, satisfying the following relationship:

$$f = \frac{1}{MN} {\sum }_{i}^{M} {\sum }_{j}^{N} {T}_{c} ( i , j ) ,$$

where MN represents a size of a feature map; and T_{c}(i,j) represents a c-th channel T_{c} at a point of (i,j).

In some embodiments, a channel attention weight q of the multi-scale feature map is calculated through two convolutional layers and sigmoid and rectified linear unit (ReLU) functions, where q satisfies the following relationship:

$$q = Sigmoid ( conv ( Relu ( conv ( f ) ) ) ) ,$$

where conv represents a convolution computation.

The multi-scale feature map is multiplied by the channel attention weight to generate an attention map. An element-wise multiplication between the attention map and the feature map is performed to obtain the image features.

According to the embodiment of the present disclosure, the method for constructing the groundwater NAPL contamination level assessment model based on deep learning and training the groundwater NAPL contamination level assessment model by utilizing the groundwater NAPL contamination database is performed through the following steps.

The groundwater NAPL contamination level assessment model is constructed based on deep learning, and the feature dataset is extracted from the groundwater NAPL contamination database.

Criteria for assessing contamination levels of each of the types corresponding to NAPL contamination are acquired in a preset retrieval space by means of big data, and a criterion with a highest occurrence frequency is selected from testing data to construct a system for an assessing system for the contamination levels of each of the types.

A contamination level assessment is performed based on the assessing system and a part of the monitoring data of the groundwater monitoring wells corresponding to the feature dataset, and contamination level annotations are set for the image features to give an annotated feature dataset.

The groundwater NAPL contamination level assessment model is trained based on the annotated feature dataset until training iterations reaches a preset value, thereby outputting a trained groundwater NAPL contamination assessment model.

The image features of the water image data and the local hydrogeological information of the research area are input into the trained groundwater NAPL contamination assessment model to identify the type and concentration information corresponding to the image features of each of the groundwater monitoring wells.

A current spatial distribution of the NAPLs and the groundwater NAPL contamination levels within the research area is acquired.

Historical monitoring data of the groundwater monitoring wells within the research area is read to determine whether each of the groundwater monitoring wells is valid, and invalid groundwater monitoring wells are removed. The monitoring data of valid groundwater monitoring wells is acquired within the preset period and performed a data cleaning to obtain the types and concentrations of the NAPLs in groundwater. The image features are integrated with the types and concentrations of the NAPLs in groundwater to construct the feature dataset. Flow direction and velocity of the groundwater, surface water-groundwater interaction frequency and a stratigraphic information in the research area are collected to construct a hydrogeological dataset, followed by combining the hydrogeological dataset with the feature dataset to generate a combined feature data. The groundwater NAPL contamination database is established. The combined feature data is structured to obtain a structured feature data. The structured feature data is stored into the groundwater NAPL contamination database. Data management of groundwater NAPL contamination in the research area is performed, and updating and optimization of the groundwater NAPL contamination level assessment model are also performed based on the groundwater NAPL contamination database.

According to the embodiment of the present disclosure, the method for issuing an early warning based on the NAPL contamination level is performed through the following steps.

Current types and concentrations of the NAPLs in each of the plurality of sub-areas are acquired based on a current spatial distribution of the NAPLs in the research area, and the current types and concentrations are correspondingly compared with historical types and concentrations of the NAPLs over a previous preset period.

If the types of the NAPLs in a sub-area change, or the concentration change of the NAPLs in a sub-area is greater than the contamination change reference value, the early warning of the sub-area is generated, and the early warning and the current spatial distribution of the NAPLs are visually displayed.

If a sub-area is not labeled, environmental features and climate features of each of the sub-area are acquired within a next preset period, and if a sub-area is labeled, environmental features, climate features and contamination source operational features of the sub-area are acquired over the next preset period, and a similarity between acquired features and the contamination impact features of each of the plurality of sub-areas is calculated based on a Manhattan distance.

If the Manhattan distance is not greater than a preset distance threshold, it is confirmed that the similarity satisfies a preset requirement, and generating a contamination early warning for a corresponding sub-area is generated, and a contamination emergency measure for the corresponding sub-area is generated and issued by means of big data based on the contamination early warning.

It should be noted that the research area is divided into a plurality of sub-areas based on distribution of the groundwater monitoring wells. Types and historical concentrations of the NAPLs in each of the plurality of sub-areas are obtained within the preset period to generate a concentration time series corresponding to the types. The contamination sources are traced from different types traced based on the concentration time series in each of the plurality of sub-areas. Each of the plurality of sub-areas contaminated by the contamination sources is marked, and is set labels according to the types of the contamination sources. A concentration change over the preset time interval is extracted based on the concentration time series of the types to set different change thresholds for labeled sub-areas and unlabeled sub-areas. It is determined whether each of the plurality of sub-areas is labeled to give a labeled sub-area or an unlabeled sub-area, followed by reading a change threshold based on a determination result.

In a case that the concentration change exceeds the change threshold, if a sub-area is not labeled, environmental features and climate features of the sub-area are acquired as contamination impact features of the sub-area; and if a sub-area is labeled, environmental features, climate features and contamination source operational features of the sub-area are acquired as the contamination impact features of the sub-area. The contamination source operational features include a production plan and production quantity of a factory, etc. Meanwhile, an average concentration change of the plurality of sub-areas over the preset time interval is acquired in the sub-area to be set as a contamination change reference value within the preset period for each of the plurality of sub-areas.

In a third aspect, the present disclosure provides a computer-readable storage medium. A computer program is stored in the computer-readable storage medium, and is configured to be executed by the processor 42 to implement any one step of the method for assessing contamination levels of non-aqueous phase liquid (NAPL) in groundwater based on image learning.

It should be understood that the devices and methods disclosed in the embodiments of the disclosure, can be implemented through other ways. The device embodiments described above are merely illustrative. For example, the division of the unit is merely a logical functional division, and also can be another way of division in actual implementation. Specifically, a plurality of units and components can be combined, or integrated into another system, and some features can be ignored, or do not be executed. Furthermore, a coupling, a direct coupling and a communication connection among the components provided herein can be achieved via a plurality of ports, and an indirect coupling or a communication connection among the devices and the units can be electrical, mechanical or of other forms.

The units described as a separate component above can be or not be physically separated, and the components shown as the unit can be or not be physical unit. The components can be arranged in a place or distributed across a plurality of network units. Some or all of the units can be selected according to practical requirements to achieve the objects of the embodiment solutions.

In addition, in the embodiments of the present disclosure, all functional units can be fully integrated into a processing unit, or each of the all functional units can be served as a separate unit, or a plurality of units can be integrated into a single unit. The abovementioned integrated unit can be configured as a hardware or a combination of a hardware and a software functional unit.

It should be understood by those of ordinary skill in the art, all or some of the steps to implement the embodiments provided herein can be accomplished through a hardware related to program instructions. The aforementioned computer program is stored in the computer-readable storage medium, and is configured to be executed to implement any one step of the embodiment. The computer-readable storage medium includes a mobile storage device, a read-only memory (ROM), a random access memory (RAM), a disk or an optical disc and other media that can store program codes.

Moreover, the integrated unit described above is configured as a software function module and an independent product for sale or use, and can be stored the computer-readable storage medium. Based on this understanding, an essence of the technical solutions of the embodiments in the present disclosure, or a part of the technical solutions of the embodiments in the present disclosure contributing to the prior art, can be configured as a software product. The software product is stored in the computer-readable storage medium, including a plurality of instructions. The plurality of instructions are configured to allow a computer device (such as a personal computer, a server, and an internet device) to execute all or some steps of the embodiments of the present disclosure. The computer-readable storage medium includes a mobile storage device, a read-only memory (ROM), a random access memory (RAM), a disk or an optical disc and other media that can store program codes.

It should be noted that the embodiments disclosed herein are merely illustrative of the disclosure, and are not intended to limit the present disclosure. Various changes, and replacements can be made by those of ordinary skill in the art to the aforementioned embodiments, and those made without departing from the spirit of the disclosure shall fall within the scope of the present disclosure defined by the appended claims.

What is claimed is:

1. A method for assessing contamination level of non-aqueous phase liquids (NAPL) in groundwater based on image learning, comprising:
(S1) acquiring water image data of groundwater monitoring wells within a research area; preprocessing the water image data; and extracting image features from a preprocessed water image data;
(S2) obtaining types and concentrations of NAPLs in groundwater based on monitoring data of the groundwater monitoring wells collected within a preset period;
(S3) matching the image features with the types and concentrations of the NAPLs to construct a feature dataset; and establishing a groundwater NAPL contamination database based on the feature dataset in combination with local hydrogeological information;
(S4) constructing a groundwater NAPL contamination level assessment model based on deep learning; training the groundwater NAPL contamination level assessment model by utilizing the groundwater NAPL contamination database; determining a NAPL contamination level of water in the groundwater monitoring wells based on a trained groundwater NAPL contamination level assessment model; and issuing an early warning based on the NAPL contamination level.

2. The method of claim 1, wherein step (S1) comprises:
obtaining, by an image sensor deployed in the groundwater monitoring wells, the water image data over a preset time interval;
subjecting the water image data to filtering for denoising, enhancement and partitioning to generate a plurality of image patches;
calculating an information entropy of each of the plurality of image patches;
generating a mask image of the water image data based on information entropies of the plurality of image patches;
performing an image segmentation on the mask image of the water image data to obtain the preprocessed water image data;
inputting the preprocessed water image data into a cross stage partial network (CSPNET) for feature characterization to generate a feature map;
introducing the feature map into an atrous spatial pyramid pooling (ASPP) module for multi-scale feature extraction by using dilated convolutions with different dilation rates to generate a multi-scale feature map;
transforming the multi-scale feature map into a channel descriptor through average pooling;
calculating a channel attention weight of the multi-scale feature map through two convolutional layers and sigmoid and rectified linear unit (ReLU) functions;
multiplying the multi-scale feature map by the channel attention weight to generate an attention map; and
performing element-wise multiplication between the attention map and the feature map to obtain the image features.

3. The method of claim 1, wherein steps (S2-S3) comprises:
reading historical monitoring data of the groundwater monitoring wells within the research area;
determining whether each of the groundwater monitoring wells is valid based on the historical monitoring data, and removing invalid groundwater monitoring wells;
acquiring monitoring data of valid groundwater monitoring wells within the preset period;
performing data cleaning on the monitoring data of the valid groundwater monitoring wells to obtain the types and concentrations of the NAPLs in groundwater;
integrating the image features with the types and concentrations of the NAPLs in groundwater to construct the feature dataset;
collecting flow direction and velocity of the groundwater, surface water-groundwater interaction frequency and a stratigraphic information in the research area to construct a hydrogeological dataset;
combining the hydrogeological dataset with the feature dataset to generate a combined feature data;
establishing the groundwater NAPL contamination database;
structurally processing and storing the combined feature data into the groundwater NAPL contamination database; and
performing data management of groundwater NAPL contamination in the research area, and updating and optimization of the groundwater NAPL contamination level assessment model based on the groundwater NAPL contamination database.

4. The method of claim 1, wherein steps of constructing the groundwater NAPL contamination level assessment model based on deep learning and training the groundwater NAPL contamination level assessment model by utilizing the groundwater NAPL contamination database comprise:
constructing the groundwater NAPL contamination level assessment model based on deep learning;
extracting the feature dataset from the groundwater NAPL contamination database;
acquiring criteria for assessing contamination levels of each of the types corresponding to NAPL contamination in a preset retrieval space by means of big data;
selecting a criterion with a highest occurrence frequency from testing data to construct an assessing system for the contamination levels of each of the types;
performing contamination level assessment based on the assessing system and a part of the monitoring data of the groundwater monitoring wells corresponding to the feature dataset;
respectively setting contamination level annotations for the image features to give an annotated feature dataset;
training the groundwater NAPL contamination level assessment model based on the annotated feature dataset until training iterations reaches a preset value, thereby outputting a trained groundwater NAPL contamination assessment model;
inputting the image features of the water image data and the local hydrogeological information of the research area into the trained groundwater NAPL contamination assessment model to identify type and concentration information corresponding to the image features of each of the groundwater monitoring wells; and
acquiring a current spatial distribution of the NAPLs and the groundwater NAPL contamination levels within the research area.

5. The method of claim 1, wherein before generating the early warning based on the groundwater NAPL contamination level, the method further comprises:
dividing the research area into a plurality of sub-areas based on distribution of the groundwater monitoring wells;
obtaining types and historical concentrations of the NAPLs in each of the plurality of sub-areas within the preset period;
generating a concentration time series corresponding to the types based on the historical concentrations;
tracing contamination sources from different types based on the concentration time series in each of the plurality of sub-areas;
marking each of the plurality of sub-areas contaminated by the contamination sources, and setting labels for the plurality of sub-areas according to the types of the contamination sources;
extracting a concentration change over the preset time interval based on the concentration time series, and setting different change thresholds for labeled sub-areas and unlabeled sub-areas;
determining whether each of the plurality of sub-areas is labeled, and reading a change threshold based on a determination result;
in a case that the concentration change exceeds the change threshold, if a sub-area is not labeled, environmental features and climate features of the sub-area are acquired as contamination impact features of the sub-area; and if a sub-area is labeled, environmental features, climate features and contamination source operational features of the sub-area are acquired as the contamination impact features of the sub-area;
acquiring an average concentration change of the plurality of sub-areas over the preset time interval; and
setting the average concentration change as a contamination change reference value within the preset period for each of the plurality of sub-areas.

6. The method of claim 5, wherein the step of generating the early warning based on the groundwater NAPL contamination level comprises:
acquiring current types and concentrations of the NAPLs in each of the plurality of sub-areas based on a current spatial distribution of the NAPLs in the research area, and correspondingly comparing the current types and concentrations with historical types and concentrations of the NAPLs over a previous preset period;
if the types of the NAPLs in a sub-area change, or the concentration change of the NAPLs in a sub-area is greater than the contamination change reference value, generating an early warning of the sub-area, and visually displaying the early warning and the current spatial distribution of the NAPLs;
if a sub-area is not labeled, acquiring environmental features and climate features of the sub-area within a next preset period; if a sub-area is labeled, acquiring environmental features, climate features and contamination source operational features of the sub-area over the next preset period; and calculating a similarity between acquired features and the contamination impact features of each of the plurality of sub-areas based on a Manhattan distance; and
if the Manhattan distance is not greater than a preset distance threshold, confirming that the similarity satisfies a preset requirement, and generating a contamination early warning for a corresponding sub-area; and generating and issuing a contamination emergency measure for the corresponding sub-area by means of big data based on the contamination early warning.

7. A system for assessing contamination levels of non-aqueous phase liquid (NAPL) in groundwater based on image learning, comprising:
a memory; and
a processor;
wherein the memory is configured to store a program; and
the processor is configured to execute the program stored in the memory to perform steps of:
(S1) acquiring water image data of groundwater monitoring wells within a research area; preprocessing the water image data; and extracting image features from a preprocessed water image data;
(S2) obtaining types and concentrations of NAPLs in groundwater based on monitoring data of the groundwater monitoring wells collected within a preset period;
(S3) matching the image features with the types and concentrations of the NAPLs to construct a feature dataset; and establishing a groundwater NAPL contamination database based on the feature dataset in combination with local hydrogeological information;
(S4) constructing a groundwater NAPL contamination level assessment model based on deep learning; training the groundwater NAPL contamination level assessment model by utilizing the groundwater NAPL contamination database; determining a NAPL contamination level of water in the groundwater monitoring wells based on a trained groundwater NAPL contamination level assessment model; and issuing an early warning based on the NAPL contamination level.

8. The system of claim 7, wherein step (S1) comprises:
obtaining, by an image sensor deployed in the groundwater monitoring wells, the water image data over a preset time interval;
subjecting the water image data to filtering for denoising, enhancement and partitioning to generate a plurality of image patches;
calculating an information entropy of each of the plurality of image patches;
generating a mask image of the water image data based on information entropies of the plurality of image patches;
performing an image segmentation on the mask image of the water image data to obtain the preprocessed water image data;
inputting the preprocessed water image data into a cross stage partial network (CSPNET) for feature characterization to generate a feature map;
introducing the feature map into an atrous spatial pyramid pooling (ASPP) module for multi-scale feature extraction by using dilated convolutions with different dilation rates to generate a multi-scale feature map;
transforming the multi-scale feature map into a channel descriptor through average pooling;
calculating a channel attention weight of the multi-scale feature map through two convolutional layers and sigmoid and rectified linear unit (ReLU) functions;
multiplying the multi-scale feature map by the channel attention weight to generate an attention map; and
performing element-wise multiplication between the attention map and the feature map to obtain the image features.

9. The system of claim 7, wherein steps (S2-S3) comprises:
reading historical monitoring data of the groundwater monitoring wells within the research area;
determining whether each of the groundwater monitoring wells is valid based on the historical monitoring data, and removing invalid groundwater monitoring wells;
acquiring monitoring data of valid groundwater monitoring wells within the preset period;
performing data cleaning on the monitoring data of the valid groundwater monitoring wells to obtain the types and concentrations of the NAPLs in groundwater;
integrating the image features with the types and concentrations of the NAPLs in groundwater to construct the feature dataset;
collecting flow direction and velocity of the groundwater, surface water-groundwater interaction frequency and a stratigraphic information in the research area to construct a hydrogeological dataset;
combining the hydrogeological dataset with the feature dataset to generate a combined feature data;
establishing the groundwater NAPL contamination database;
structurally processing and storing the combined feature data into the groundwater NAPL contamination database; and
performing data management of groundwater NAPL contamination in the research area, and updating and optimization of the groundwater NAPL contamination level assessment model based on the groundwater NAPL contamination database.

10. The system of claim 7, wherein steps of constructing the groundwater NAPL contamination level assessment model based on deep learning and training the groundwater NAPL contamination level assessment model by utilizing the groundwater NAPL contamination database comprise:
constructing the groundwater NAPL contamination level assessment model based on deep learning;
extracting the feature dataset from the groundwater NAPL contamination database;
acquiring criteria for assessing contamination levels of each of the types corresponding to NAPL contamination in a preset retrieval space by means of big data;
selecting a criterion with a highest occurrence frequency from testing data to construct an assessing system for the contamination levels of each of the types;
performing contamination level assessment based on the assessing system and a part of the monitoring data of the groundwater monitoring wells corresponding to the feature dataset;
respectively setting contamination level annotations for the image features to give an annotated feature dataset;
training the groundwater NAPL contamination level assessment model based on the annotated feature dataset until training iterations reaches a preset value, thereby outputting a trained groundwater NAPL contamination assessment model;
inputting the image features of the water image data and the local hydrogeological information of the research area into the trained groundwater NAPL contamination assessment model to identify type and concentration information corresponding to the image features of each of the groundwater monitoring wells; and
acquiring a current spatial distribution of the NAPLs and the groundwater NAPL contamination levels within the research area.
